(19) United States
(12) Patent Application Publication
(10) Pub. No.: US 2020/0297317 A1
(43) Pub. Date: Sep. 24, 2020

(54) ULTRASONIC DIAGNOSTIC DEVICE AND TRANSMISSION CONTROL METHOD

(71) Applicant: Hitachi, Ltd., Tokyo (JP)

(72) Inventor: Shinta Takano, Tokyo (JP)

(73) Assignee: Hitachi, Ltd., Tokyo (JP)

(21) Appl. No.: 16/646,634

(22) PCT Filed: Dec. 26, 2018
(86) PCT No.: PCT/JP2018/047883

§ 371 Date: Mar. 12, 2020

(30) Foreign Application Priority Data
Mar. 16, 2018 (JP) 2018-049202

(51) Int. Cl. A61B 8/00; A61B 8/08

(52) U.S. Cl. CPC A61B 8/4494; A61B 8/54; A61B 8/0866

(57) ABSTRACT

A plurality of opening positions is determined along a θ direction which is a curved direction on a two-dimensional transducer element array. Transmission openings are sequentially set to the plurality of opening positions. Transmission beam deflection scanning is performed at each opening position using the transmission openings. Transmission beam lines radiating from the center of the transmission opening are thus formed. A transmission apodization is applied before and after switching from one opening position to another so that there are no steps in the transmission sound field.

### TECHNICAL FIELD

The present invention relates to an ultrasonic diagnostic device and a transmission control method, and particularly, to transmission control of an ultrasonic diagnostic device having a two-dimensional transducer element array.

### BACKGROUND

Ultrasonic diagnostic devices for performing three-dimensional ultrasonic diagnosis have been spreading. In such ultrasonic diagnostic devices, 3D probes are used. In general, such a 3D probe has a two-dimensional transducer element array and an electronic circuit. The two-dimensional transducer element array is composed of hundreds, thousands, tens of thousands, or more transducer elements arranged two-dimensionally. The electronic circuit is a circuit for supplying a plurality of element transmission signals to the two-dimensional transducer element array and processing a plurality of element reception signals received from the two-dimensional transducer element array.

Specifically, during transmission, with respect to each transmission signal output from the main device body of the ultrasonic diagnostic device, the electronic circuit generates a plurality of element transmission signals on the basis of the corresponding transmission signal by performing a delay process, and outputs them to a sub-array (a plurality of transducer elements constituting transducer element group) in parallel. Meanwhile, during reception, with respect to each sub-array, the electronic circuit performs delay and addition processes on a plurality of element reception signals output in parallel from the corresponding sub-array, thereby generating a reception signal. Such signal processing which is performed in sub-array units is called sub beamforming. In the main device body, a plurality of transmission signals are generated by a delay process, and they are output to the electronic circuit provided in the 3D probe. Also, in the main device body, delay and addition processes are further performed on a plurality of reception signals output from the electronic circuit provided in the 3D probe, whereby beam data are generated. Such signal processing which is performed on all of the plurality of sub-arrays is called main beamforming. The electronic circuit provided in the 3D probe is a circuit for channel reduction.

Patent Document 1 and Patent Document 2 disclose ultrasonic diagnostic devices having a plurality of sub-beamformers (a plurality of micro-beamformers) and main beamformers. Patent Document 3 discloses an ultrasonic diagnostic device having a 1D transducer element array. Those ultrasonic diagnostic devices use apodization curves (weighting functions) to form reception beams.

### CITATION LIST

### Patent Literature

Patent Document 1: JP 5572633 B

Patent Document 2: JP 2005-270423 A

Patent Document 3: JP 4717109 B

### SUMMARY

### Technical Problem

In the case of using a 3D probe, if every control for transmitting and receiving ultrasonic waves is performed in transducer element units, the amount of control data which need to be handled and the amount of control data which need to be transmitted increase, making real-time control difficult. As the number of transducer elements increases, the above-mentioned problem becomes more remarkable. Meanwhile, if the amount of control is simply reduced, ultrasonic images degrade in quality.

An object of the present disclosure is to reduce the amount of control for transmission control in an ultrasonic diagnostic device having a 3D probe. Another object of the present disclosure is to reduce the amount of control for transmission control while maintaining or improving the qualities of ultrasonic images, in an ultrasonic diagnostic device having a 3D probe.

### Solution to Problem

An ultrasonic diagnostic device according to the present disclosure is characterized by including a two-dimensional transducer element array that is composed of a plurality of sub-arrays arranged two-dimensionally, an electronic circuit that is connected to the two-dimensional transducer element array and performs signal processing in sub-array units, and a system control unit that controls transmission and reception of ultrasonic waves by controlling the electronic circuit, wherein by controlling the electronic circuit, a plurality of opening positions are determined on the two-dimensional transducer element array, with a sub-array pitch along a scanning direction, and at the plurality of opening positions, two-dimensional transmission openings which are sub-array sets are sequentially set, and at each of the opening positions, transmission beam deflection scanning in the scanning direction is performed.

A transmission control method according to the present disclosure is characterized by controlling an electronic circuit connected to a two-dimensional transducer element array composed of a plurality of sub-arrays arranged two-dimensionally such that a plurality of opening positions are determined on the two-dimensional transducer element array, with a sub-array pitch along a scanning direction, and at the plurality of opening positions, two-dimensional transmission openings which are sub-array sets are sequentially set, and at each of the opening positions, transmission beam deflection scanning in the scanning direction is performed.

### BRIEF DESCRIPTION OF DRAWINGS

FIG. 1 is a block diagram illustrating an ultrasonic diagnostic device according to an embodiment.

FIG. 2 is a block diagram illustrating a transceiver.

FIG. 3 is a circuit diagram illustrating a transmission voltage generation circuit.

FIG. 4 is a view illustrating a convex 3D probe.

FIG. 5 is a view illustrating a first example of transmission openings.

FIG. 6 is a view illustrating a second example of the transmission openings.

FIG. 7 is a view illustrating a third example of the transmission openings.

FIG. 8 is a view illustrating a fourth example of the transmission openings.

FIG. 9 is a view illustrating beam profiles.

FIG. 10 is a view illustrating transmission beam deflection scanning which is repeatedly performed in a scanning procedure of the transmission openings.

FIG. 11 is a view illustrating the relation between a scanning line array and a transmission beam array.

FIG. 12 is a view illustrating two neighboring scanning line arrays.

FIG. 13 is a view illustrating two neighboring transmission beam arrays.

FIG. 14 is a view illustrating characteristics of a left-end transmission beam.

FIG. 15 is a view illustrating characteristics of a right-end transmission

FIG. 16 is a view illustrating switching of transmission apodization curves.

FIG. 17 is a view illustrating application of the same transmission apodization curve to a plurality of transducer element rows.

FIG. 18 is a view illustrating two transmission apodization curves which can be applied before and after switching from one opening position to another.

FIG. 19 is a view illustrating a modification.

### DESCRIPTION OF EMBODIMENTS

Hereinafter, an embodiment will be described on the basis of the drawings.

(1) Outline of Embodiment

An ultrasonic diagnostic device according to an embodiment includes a two-dimensional transducer element array, an electronic circuit, and a system controller. The two-dimensional transducer element array is composed of a plurality of sub-arrays arranged two-dimensionally. The electronic circuit is a circuit connected to the two-dimensional transducer element array, and is a circuit for performing signal processing in sub-array units for channel reduction. The system controller is a controller for controlling the electronic circuit, thereby controlling transmission and reception of ultrasonic waves. By the control of the system controller, on the two-dimensional transducer element array, a plurality of opening positions are determined with a sub-array pitch along a scanning direction, and at the plurality of opening positions, two-dimensional transmission openings which are sub-array sets are sequentially set, and at each opening position, transmission beam deflection scanning in the scanning direction is performed.

According to the above-described configuration, since the two-dimensional transmission openings are configured in sub-array units, rather than in transducer element units, and the plurality of opening positions are determined with the sub-array pitch, rather than with a transducer element pitch, it is possible to reduce the amount of control in scanning of the two-dimensional transmission openings. Therefore, various advantages such as simplification of control, an increase in the control speed, a reduction in the size of the electronic circuit, a decrease in the power consumption of the electronic circuit, and a reduction in the cost are obtained.

Also, according to the above-described configuration, even if the plurality of opening positions are discretely set, since transmission beam deflection scanning in the scanning direction is performed at each opening position, it is possible to avoid a decrease in scanning line density, or it is possible to realize a desired scanning line density. Therefore, it is possible to prevent the quality determination of ultrasonic images, or it is possible to improve the quality of ultrasonic images. In the embodiment, transmission beam deflection scanning is performed in the scanning direction along which the plurality of opening positions have been determined and in a direction orthogonal to the scanning direction. In other words, each transmission opening is two-dimensionally sector-scanned with a transmission beam. In this case, the scanning direction can be referred to as a main scanning direction or a first scanning direction, and the direction orthogonal thereto can be referred to as a sub scanning direction or a second scanning direction.

In the embodiment, the two-dimensional transducer element array and the electronic circuit are provided inside a probe head. The system controller is provided inside the main device body. Channel reduction is for achieving a reduction in the number of channels; i.e., the number of signal lines. Herein, channel reduction means at least reception channel reduction. The sub-array pitch corresponds to the sub-array length in the scanning direction. According to transmission beam deflection scanning, even if the sub-array pitch is set to be large, it is possible to realize a desired scanning line density. Here, in the embodiment, each scanning line corresponds to a reception scanning line to which dynamic focusing for reception is applied, in the case where parallel reception is not performed, and corresponds to the center line of a plurality of reception scanning lines having a parallel reception relation, in the case where parallel reception is performed. The above-described configuration is the realization of a combination of electronic scanning of transmission opening with the sub-array pitch and electronic sector-scanning of transmission beam, which is performed in transmission opening units, in the scanning direction.

In the embodiment, the two-dimensional transducer element array is composed of a plurality of transducer elements arranged two-dimensionally along a convex surface having a curvature direction which is the scanning direction and a width direction orthogonal to the curvature direction, and a two-dimensional transmission opening is scanned in the curvature direction. The convex surface of the convex 3D probe is a relatively wide surface extending in the scanning direction, and it is necessary to dispose a number of transducer elements on the convex surface. In this case, it is especially demanded to reduce the amount of control. The above-described configuration is suitable for such a demand.

In the embodiment, each sub-array has a longitudinal direction parallel with the curvature direction and a transverse direction parallel with the width direction, and in each sub-array, the number of transducer elements in the longitudinal direction is greater than the number of transducer elements in the transverse direction. According to this configuration, it is possible to reduce the number of sub-arrays in the curvature direction, thereby reducing the amount of control.

In the embodiment, at each opening position a plurality of scanning lines which spread out radially from an origin point are set, at each opening position a plurality of transmission beams which spread out radially from the center of a two-dimensional transmission opening are formed, and on the plurality of scanning lines a plurality of transmission focuses are formed. The above-mentioned origin point is a predetermined point from which the plurality of scanning lines are projected, and is generally the origin point for reception scanning. For example, the center of curvature of the convex surface may be set as the origin point, or any other point may be set as the origin point. In the embodiment, according to the deflection angle of each transmission beam, a transmission apodization curve to be used is selected from a transmission apodization curve array. Therefore, it is possible to improve the quality of ultrasonic images. Transmission apodization curves are preferably curves which are weighted in transducer element units, rather than in sub-array units.

Scanning of the transmission opening is rough control which can be performed with the sub-array pitch; whereas transmission beam deflection scanning and transmission apodization are minute control which can be performed in transducer element units. The above-described configuration realizes a combination of rough control and minute control.

In the embodiment, the transmission apodization curve array is commonly used for the plurality of opening positions. Therefore, it is possible to restrain an increase in the amount of control caused by performing transmission apodization.

In the embodiment, each transmission apodization curve has a form for making the peak of the profile of each transmission beam coincide with each scanning line in the front and rear of a transmission focus on the corresponding scanning line. According to this configuration, it becomes difficult for steps to occur in a transmission sound field before and after switching from an opening position to another. Such steps become factors which cause vertical stripe patterns on ultrasonic images, and according to the above-described configuration, it is possible to suppress or prevent occurrence of vertical stripe patterns.

In the embodiment, the two-dimensional transmission opening is composed of a plurality of transducer element rows arranged in an orthogonal direction orthogonal to the scanning direction, each transducer element row is composed of a plurality of transducer elements arranged in the scanning direction, and each transmission apodization curve is commonly applied to the plurality of transducer element rows arranged in the orthogonal direction. According to this configuration, as compared with the case of applying different transmission apodization curves to the transducer element rows, respectively, it is possible to significantly reduce the amount of control.

In the embodiment, the electronic circuit includes a plurality of transceivers connected to the plurality of transducer elements constituting the two-dimensional transducer element array, each transceiver includes a transmission voltage generation circuit for generating transmission voltage which is defined by a transmission apodization curve which is used, each transmission voltage generation circuit generates transmission voltage by dividing maximum transmission voltage, and a voltage control value standardized according to the maximum transmission voltage is given to each transmission voltage generation circuit. According to this configuration, as compared with the case of indicating a specific voltage value, it is possible to reduce control data.

In the embodiment, the shape of the two-dimensional transmission opening which is set at the individual opening positions is a polygonal shape which is formed by cutting off four corners from a rectangular shape extending in the curvature direction, or an ellipsoidal shape extending in the curvature direction. According to this configuration, it is possible to reduce side lobes. The size or shape of the two-dimensional transmission opening may be changed according to transmission focus depth. If the shape of the two-dimensional transmission opening is maintained during scanning of the two-dimensional transmission opening, it is possible to reduce the amount of control.

In the embodiment, during transmission beam deflection scanning at each opening position, in the two-dimensional transmission opening, a transmission apodization curve is scanned in the scanning direction while the shape thereof is maintained. If the transmission apodization curve defining an effective opening is electronically scanned in the transmission opening, it is possible to restrain or prevent steps from occurring in a transmission sound field before and after switching from an opening position to another.

(2) Details of Embodiment

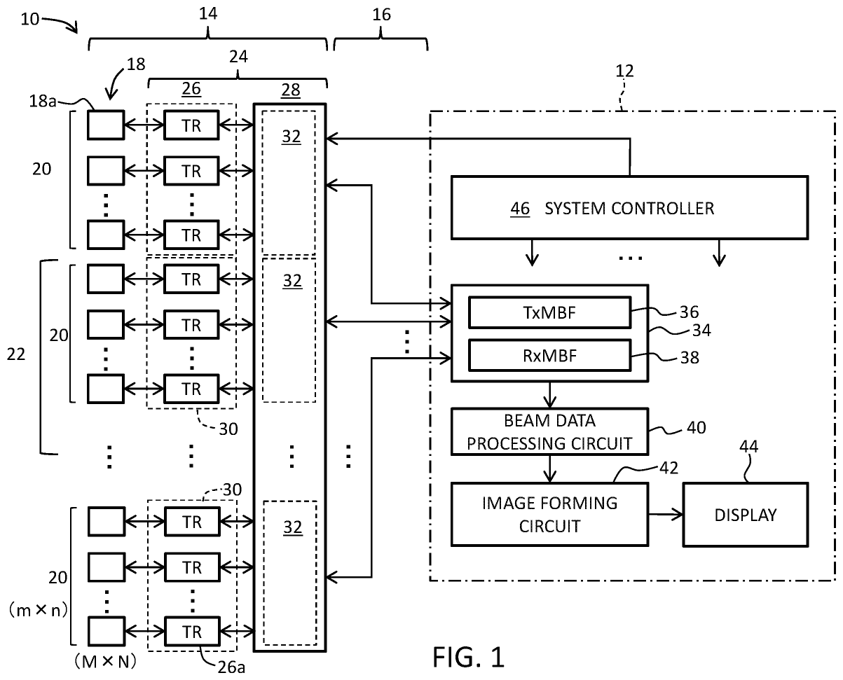

FIG. 1 shows the ultrasonic diagnostic device according to the embodiment. This ultrasonic diagnostic device is generally installed in a medical institution, and is a device for forming ultrasonic images for diagnosis on the basis of reception data obtained by transmitting and receiving ultrasonic waves to and from subjects (biological bodies). The ultrasonic diagnostic device according to the embodiment has a function of performing two-dimensional scanning with ultrasonic beam, thereby acquiring volume data, and forming a three-dimensional ultrasonic image on the basis of the volume data. Hereinafter, a detailed description thereof will be made.

In FIG. 1, the ultrasonic diagnostic device includes a probe 10 and a main device body 12. The probe 10 is a so-called 3D probe, and is configured with a probe head 14, a cable 16, and a connector (not shown in the drawings). The connector is connected to the main device body 12 so as to be removable. The probe head 14 is a portable wave transceiver which can be held by a user (such as a doctor or a laboratory technician). The wave transmission/reception surface of the probe head 14 is put on the surface of a body, and in this state, an ultrasonic wave is transmitted and received. The probe 10 according to the embodiment is a 3D probe which can be used in obstetrics departments in order to perform three-dimensional diagnosis on fetuses, and the wave transmission/reception surface thereof constitutes the convex surface (the convex surface having a cylindrical surface). In other words, the probe 10 is a convex 3D probe. 3D probes having flat wave transmission/reception surfaces, 3D probes for insertion in body cavities, and the like may be used.

In the probe head 14, a two-dimensional transducer element array 18 and an electronic circuit 24 are disposed. The two-dimensional transducer element array 18 is an array composed of a plurality of transducer elements 18a two-dimensionally arranged along the convex surface. The number of transducer elements 18a is M×N; for example, tens of thousands. The two-dimensional transducer element array 18 is composed of a plurality of sub-arrays 20. In other words, the two-dimensional transducer element array 18 is divided into the plurality of sub-arrays 20 for transmission/reception control. Specifically, in the two-dimensional transducer element array 18, the plurality of sub-arrays 20 arranged two-dimensionally are set. The number of sub-arrays 20 is m×n; for example, several hundreds. Each sub-array 20 is composed of, for example, about tens of or one hundred transducer elements grouped for channel reduction. However, all of numeric values which are disclosed in this specification are merely illustrative.

On the two-dimensional transducer element array 18, a transmission opening 22 is set. The transmission opening 22 is a two-dimensional transmission opening, and it corresponds to a sub-array set. In other words, the transmission opening is composed of a plurality of sub-arrays 20 arranged two-dimensionally. In other words, the transmission opening 22 is configured using the sub-arrays 20 as units. As will be described below, along the scanning direction which is the curvature direction, a plurality of opening positions are set with the sub-array pitch, and the transmission openings 22 are sequentially set at the plurality of opening positions. As described above, the transmission opening 22 is configured in sub-array units, and the transmission opening 22 shift stepwise in sub-array units. Therefore, during setting and control of the transmission opening 22, it is possible to significantly reduce the amount of control (the amount of control data, the amount of transmission data, and the like).

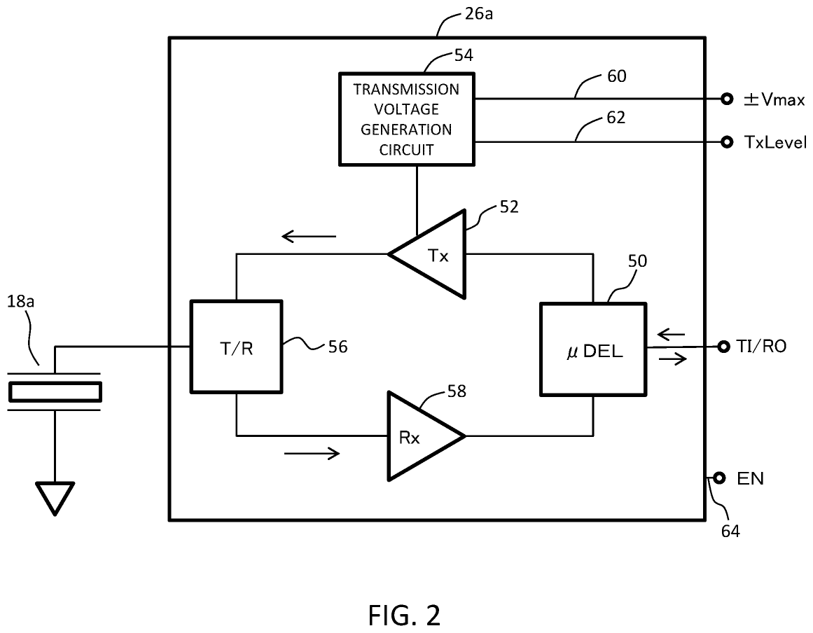

The electronic circuit 24 is connected to the two-dimensional transducer element array 18. The electronic circuit 24 includes a transceiver array 26 and a processing circuit 28. The processing circuit 28 has a signal processing function and a control function. When attention is paid to the relation between the two-dimensional transducer element array 18 and the electronic circuit 24, one transceiver 26a is connected to one transducer element 18a. During transmission, each of the transceivers 26a generates an element transmission signal by performing a delay process, and outputs the element transmission signal to a transducer element 18a connected to the corresponding transceiver. During reception, each transceiver performs a delay process on an element transmission signal received from a transducer element 18a connected to the corresponding transceiver. A specific example thereof will be described below with reference to FIG. 2. The transceiver array 26 is divided into groups in sub-array units for control or signal processing. In other words, a plurality of transceiver groups 30 corresponding to the plurality of sub-arrays are configured.

The processing circuit 28 is connected to the plurality of transceiver groups 30 which constitute the transceiver array 26. In the configuration example illustrated in the drawing, the processing circuit 28 includes a plurality of processing modules 32 corresponding to the plurality of transceiver groups 30. During transmission, the individual processing modules 32 output transmission signals received from the main device body 12 to the plurality of transceivers 26a connected to the processing modules, in parallel. This process is for transmission channel reduction. During reception, each of the processing modules 32 performs an addition process on a plurality of element reception signals output in parallel from a transceiver group 30 connected to the corresponding processing module and subjected to a delay process, thereby generating a reception signal (a group reception signal). The delay process and the addition process also are referred to collectively as a delay/addition process or a phasing/addition process. The plurality of reception signals generated in the plurality of processing modules 32 are output to the main device body 12, in parallel. This process is for reception channel reduction. A combination of one transceiver group 30 and one processing module 32 corresponds to one sub-beamformer. From this point of view, the electronic circuit 24 is a circuit serving as a plurality of sub-beamformers connected to the plurality of sub-arrays 20.

However, the electronic circuit 24 can have a configuration other than the above-described configuration, so long as the electronic circuit can perform transmission signal processing and reception signal processing for channel reduction. The electronic circuit 24 is actually configured with, for example, six or eight ICs. In order to suppress a rise in the temperature of the electronic circuit 24, it is desirable to configure the probe 10 as a water-cooled probe.

The main device body 12 includes a beamformer 34 which constitutes a transmitting/receiving unit. In the configuration example shown in the drawing, the beamformer 34 includes a main transmission beamformer 36 and a main reception beamformer 38. The main transmission beamformer 36 is a circuit for outputting a plurality of transmission signals obtained by applying a delay process to the electronic circuit 24 in parallel during transmission. In general, one transmission signal corresponds to one sub-array 20. The main reception beamformer 38 is a circuit for applying a delay/addition (phasing/addition) process to a plurality of reception signals (group reception signals) output in parallel from the electronic circuit 24, thereby generating beam data. One beam data item corresponds to one reception scanning line. One beam data item is composed of a plurality of echo data items arranged in the depth direction. The main transmission beamformer 36 may be provided inside the probe head 14.

A beam data processing circuit 40 is a circuit for applying wave detection, logarithmic conversion, and other signal processing to beam data. The beam data subjected to signal processing are input to an image forming circuit 42. The image forming circuit 42 is a circuit for forming a three-dimensional ultrasonic image on the basis of a plurality of beam data items (volume data items) obtained from a three-dimensional space in a biological body. On the occasion of forming a three-dimensional ultrasonic image, a well-known algorithm such as volume rendering can be used. In the image forming circuit 42, tomographic images or other images may be formed. A display 44 is configured with an LCD, an organic EL device, or the like, and on the screen of the display, ultrasonic images can be displayed.

A system controller 46 is a controller for controlling operations of individual components constituting the ultrasonic diagnostic device, and is configured with a CPU and an operation program. The system controller 46 has a transmission/reception control function. Specifically, the system controller 46 controls transmission beam scanning, reception beam scanning, transmission opening scanning, and reception opening scanning through control of the electronic circuit 24. Also, the system controller controls transmission apodization and reception apodization.

In FIG. 2, a configuration example of the transceivers 26a is shown. A transmission signal TI from the processing circuit shown in FIG. 1 is delayed by a delay element (μDEL) 50, and undergoes power amplification in a power amplifier 52, thereby becoming an element transmission signal. This element transmission signal is supplied to a transducer element 18a through a transmission/reception switch 56. If an echo from the inside of a biological body is received by the transducer element 18a, an element reception signal is generated in the transducer element 18a, and the element reception signal is input to a reception amplifier 58 through the transmission/reception switch 56, and is amplified therein, and is delayed by the delay element 50. A reception signal RO obtained by the delay process is output to the processing circuit shown in FIG. 1.

To the power amplifier 52, transmission voltage generated by a transmission voltage generation circuit 54 is applied. Reference symbol 60 indicates maximum transmission voltage (±Vmax) which can be supplied from the main device body side. The maximum transmission voltage can be changed on the main device body side. Reference symbol 62 indicates a designation value (relative value) of transmission voltage to be described below. For each sub-array, an enable signal (EN) 64 is generated. According to whether the enable signal is supplied, the operation of each of the transceivers 26a constituting the corresponding sub-array is controlled to be turned on and off. However, the transmission voltage generation circuit may be provided inside the transceiver 26a. In this case, the transmission voltage generation circuit may be provided in place of the above-mentioned power amplifier 52.

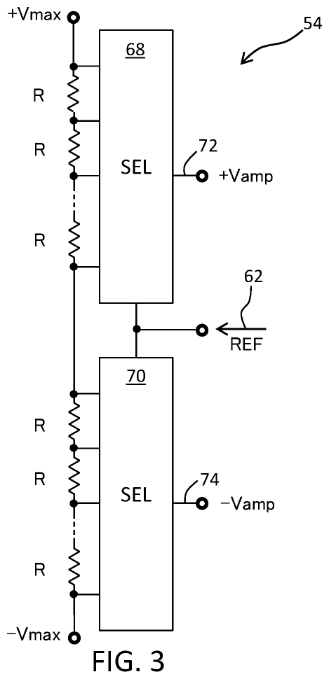

In FIG. 3, a configuration example of the transmission voltage generation circuit 54 is shown. Between the positive-side voltage +Vmax and the negative-side voltage −Vmax, a plurality of resistors R for voltage division are connected in series. To a plurality of voltage division points on the positive side (specifically, voltage extraction points in sixteen stages), a selector 68 is connected, and to a plurality of voltage extraction points on the negative side, a selector 70 is connected. The selectors 68 and 70 are for selecting any one transmission voltage pair on the basis of a command (REF) 62 designating transmission voltage. The selected positive-side transmission voltage is denoted by reference symbol 72, and the selected negative-side transmission voltage is denoted by reference symbol 74. These voltages are given to the power amplifier shown in FIG. 2, and accordingly, positive-side amplification and negative-side amplification on an element transmission signal are defined.

In the embodiment, for the transmission voltage generation circuit 54, relative values to the maximum voltages ±Vmax; i.e., standardized values, are designated, rather than actual specific voltage values. Specifically, the number of a stage selected from the sixteen stages is designated. Therefore, it is possible to reduce the amount of control data. For example, in order to specifically designate transmission voltage, it is necessary to constitute voltage command data of eight bits. According to the configuration of the embodiment, since voltage command data need only to designate the number of a stage, the voltage command data can be constituted of four bits. A configuration other than the circuit configuration shown in FIG. 3 may be used. A system or the like for changing voltage according to current control may be used.

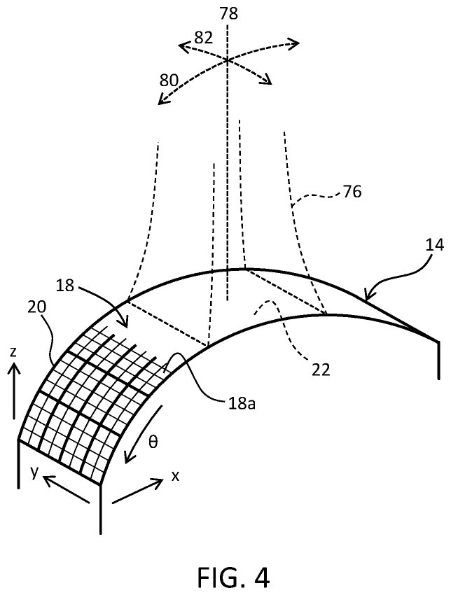

In FIG. 4, the probe head 14 of the 3D probe is shown. The two-dimensional transducer element array 18 is provided along the convex surface. As described above, the two-dimensional transducer element array 18 is composed of a number of transducer elements 18a arranged two-dimensionally. In FIG. 4, a θ direction is the curvature direction, which is the scanning direction (the opening scanning direction). A direction orthogonal to the θ direction is a y direction. The y direction is the width direction which is a horizontal direction. As another horizontal direction orthogonal to the y direction, an x direction is shown, and as a vertical direction orthogonal to the two horizontal directions, a z direction is shown.

The two-dimensional transducer element array 18 is divided into the plurality of sub-arrays 20 arranged two-dimensionally. Each of the sub-arrays 20 is an array constituting one processing unit for channel reduction as described above. On the two-dimensional transducer element array 18, the transmission openings 22 are set. In FIG. 4, for explanation, at the center in the θ direction, a transmission opening 22 is set. The width of the transmission opening 22 in the y direction extends over the whole of the two-dimensional transducer element array 18 in the y direction. A central axis 78 of the transmission opening 22 shown in FIG. 4 is parallel with a z axis.

By the transmission opening 22, a transmission beam 76 is formed along the central axis 78. As shown by reference symbol 80, in a state where the transmission opening 22 is fixed, transmission beam deflection scanning (i.e. electronic sector scanning of a transmission beam) is performed in the θ direction, whereby the θ direction is scanned with the transmission beam 76. Also, as shown by reference symbol 82, in the state where the transmission opening 22 is fixed, transmission beam deflection scanning is performed in the direction orthogonal to the θ direction, whereby the corresponding direction is scanned with the transmission beam 76.

The transmission opening 22 is intermittently scanned in the θ direction, using the length of a sub-array 20 in the θ direction as one shift unit. This is also called channel rotation. Each channel in that case corresponds to a sub-array. In other words, the distance (pitch) between two neighboring opening positions corresponds to a sub-array 20. Specifically, the plurality of opening positions are set with the sub-array pitch in the direction, and at the individual opening positions, the transmission openings 22 are sequentially set. With this, the center point of the transmission opening 22 (a base point for beam deflection scanning) sequentially shift in the θ direction.

By scanning of the transmission opening 22 in the θ direction, the transmission beam deflection scanning in the θ direction, and the transmission beam deflection scanning in the direction orthogonal to the θ direction, described above, the transmission beam is two-dimensionally scanned. In FIG. 4, reception openings and reception beams are not shown. The reception opening may be scanned similarly to the transmission opening or the reception opening may be electronically and linearly scanned with a transducer element pitch. Also, on the occasion of scanning of a reception beam, various scanning methods can be applied. During reception, parallel reception may be applied.

In FIG. 5, a first example of the transmission openings is shown. In the first example, a transmission opening 22 has a rectangular (oblong) form having the θ direction as its longitudinal direction and having the y direction as its transverse direction. The transmission opening 22 extends over the whole area in the y direction. Each sub-array 20 has a rectangular form having the θ direction as its longitudinal direction and having the y direction as its transverse direction. In each sub-array 20, the number of elements in the direction is greater than the number of elements in the y direction. The next transmission opening is denoted by reference symbol 22A. A shift amount 84 of the transmission opening 22 corresponds to the length of a sub-array 20 in the longitudinal direction.

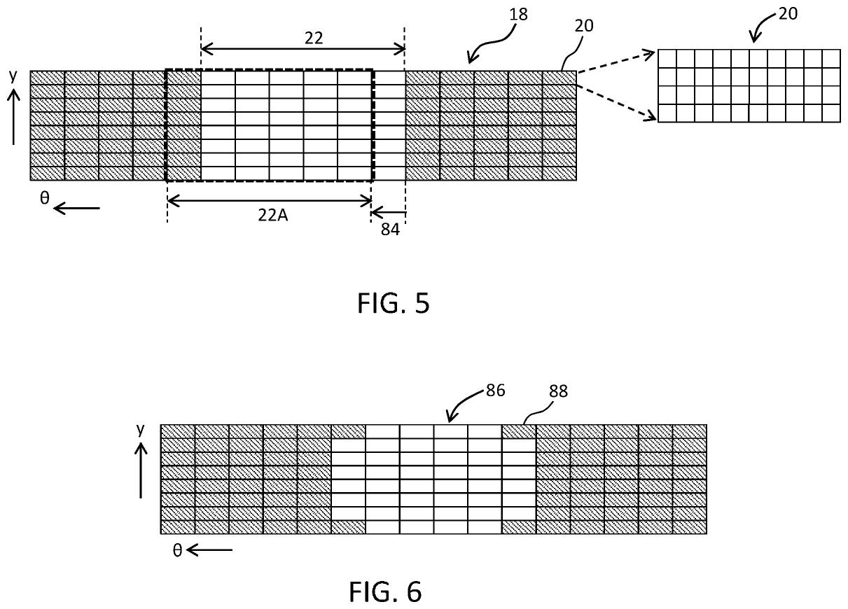

In FIG. 6, a second example of the transmission openings is shown. In the second example, a transmission opening 86 has a substantially rectangular form extending in the direction. Specifically, it has four invalid sub-arrays 88 at four corners. As a result, the form of the transmission opening 86 is close to a polygonal shape or an ellipsoidal shape. The width of the transmission opening 86 in the y direction extends over the whole area of the two-dimensional transducer element array 18 in they direction. This is the same even in a third example and a fourth example to be described below.

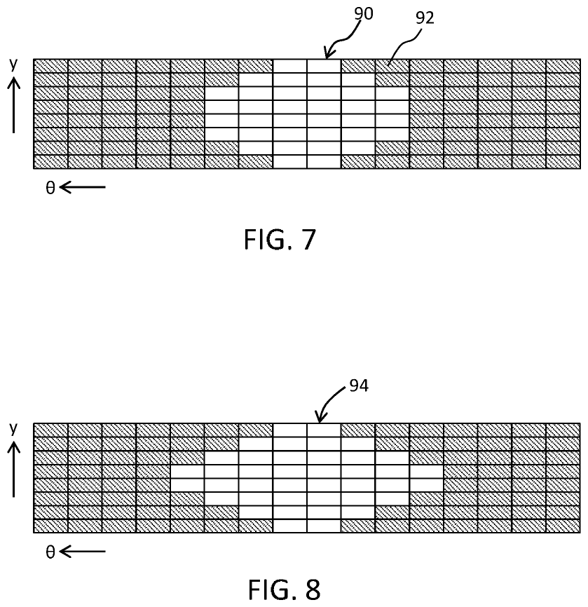

In FIG. 7, a third example of the transmission openings is shown. In the third example, a transmission opening 90 has a polygonal form extending in the θ direction. This is a form obtained by cutting off four corners from a rectangular shape, and is an ellipsoidal shape. Incidentally, in the case where a transmission opening has a polygonal shape or an ellipsoidal shape, the width of the transmission opening in the θ direction is defined as a maximum value, and the width of the transmission opening in the y direction is also defined as a maximum value.

In FIG. 8, a fourth example of the transmission openings is shown. In the fourth example, a transmission opening 94 extends in the θ direction, and has a shape close to a rhombic shape. This also is a form obtained by cutting off four corners from a rectangular shape, and can be referred to as an ellipsoidal shape. In the case where a polygonal or ellipsoidal form is used, instead of a rectangular form, as the form of a transmission opening, it is possible to reduce side lobes.

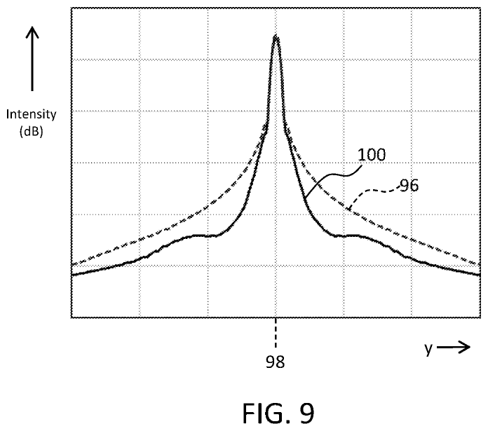

In FIG. 9, transmission beam profiles 96 and 100 in the transverse direction (the y direction) are shown. A horizontal axis indicates the y direction, and a vertical axis indicates intensity. Reference symbol 98 indicates a beam center position. The transmission beam profile 96 shows the form of a transmission beam which is formed by a rectangular transmission opening. The transmission beam profile 100 shows the form of a transmission beam which is formed by a transmission opening having a shape obtained by excluding four corner parts from the rectangular shape. As shown in FIG. 9, by approximating the form of the transmission opening to a polygonal shape or an ellipse, it is possible to reduce side lobes. When transmission apodization is omitted with respect to the transverse direction while such a transmission opening is used, an advantage of reducing the circuit scale and the amount of control while reducing side lobes is obtained.

Now, the transmission control according to the embodiment; i.e. transmission opening control and transmission beam scanning control, will be described in detail. All of the above-mentioned control is applied to the θ direction.

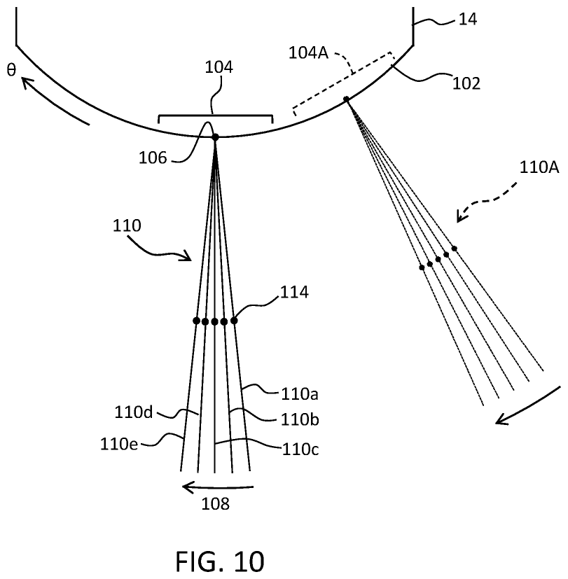

As shown in FIG. 10, in the embodiment, along the θ direction which is the scanning direction, a plurality of opening positions are set with the sub-array pitch, and the transmission openings are sequentially set at the individual opening positions. At each opening position, transmission beam deflection scanning is performed by a transmission opening set there. In FIG. 10, reference symbol 102 indicates the convex surface of the probe head 14, which corresponds to the two-dimensional transducer element array.

Reference symbol 104 indicates a transmission opening set at the middle point in the θ direction. In a state where the transmission opening 104 is fixed, beam deflection scanning 108 in the θ direction is performed, whereby a transmission beam array 110 is formed. In the example shown in the drawing, the transmission beam array 110 is composed of five transmission beams 110a to 110e spreading out radially from the center 106 of the transmission opening 104. Reference symbol 114 indicates a transmission focus array. In FIG. 10, a transmission opening 104A set at another opening position is also shown. Even at that opening position, transmission beam deflection scanning is performed, whereby a transmission beam array 110A is formed. Also, at the other openings, the same transmission beam deflection scanning is performed.

In the transmission control method according to the embodiment, when k is 1, 2, 3, etc., in a first step, a k-th transmission opening is set, and in a second step, k-th transmission beam deflection scanning is performed using the k-th transmission opening. Subsequently, in the case where it is determined in a third step that k has not reached the maximum value, in a fourth step, k is increased by 1, and the first step and the second step are performed again. Until it is determined in the fourth step that k has reached the maximum value, the series of steps described above is repeatedly performed. Thereafter, if necessary, k is initialized, and the above-mentioned transmission control method is performed again.

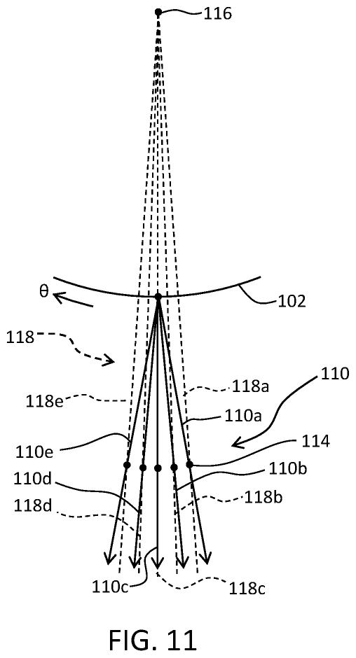

In FIG. 11, the relation between a scanning line array 118 and a transmission beam array 110. Reference symbol 116 indicates the origin point which is the center of curvature of the convex surface 102. In the example shown in the drawing, the scanning line array 118 is composed of five scanning lines 118a to 118e spreading out radially from the origin point 116. Here, each of the individual scanning lines 118a to 118e corresponds to a reception scanning line to which dynamic focusing for reception is applied in the case where parallel reception is not performed, and corresponds to the center line of an array of parallel reception scanning lines in the case where parallel reception is performed. A point other than the center of curvature may be set as the origin point 116.

On the occasion of transmission beam deflection scanning, five transmission beams 110a to 110e are sequentially formed such that transmission focuses are formed on the individual scanning lines 118a to 118e. In the case where it is desired to increase the scanning line density, more scanning lines may be set per one opening position, such that more transmission beams are formed. In the case where it is desired to perform transmission and reception with respect to a scanning line adjacent to the left of the scanning line 118e, the transmission opening is shifted by one pitch in the θ direction, and at the shifted opening position, transmission beam deflection scanning is performed.

By performing transmission beam deflection scanning at each of the plurality of opening positions set along the θ direction, echo data are acquired over the whole range or designated range in the θ direction. Incidentally, during acquisition of volume data, at each opening position, transmission beam deflection scanning is performed even in the direction orthogonal to the θ direction.

As described above, even if the transmission opening is shifted stepwise, since transmission beam deflection scanning is performed at each opening position, it is possible to realize a necessary scanning line density in the θ direction. In other words, it is possible to prevent the qualities of ultrasonic image from deteriorating or to improve the quality of ultrasonic images while reducing the amount of control for transmission beam deflection scanning.

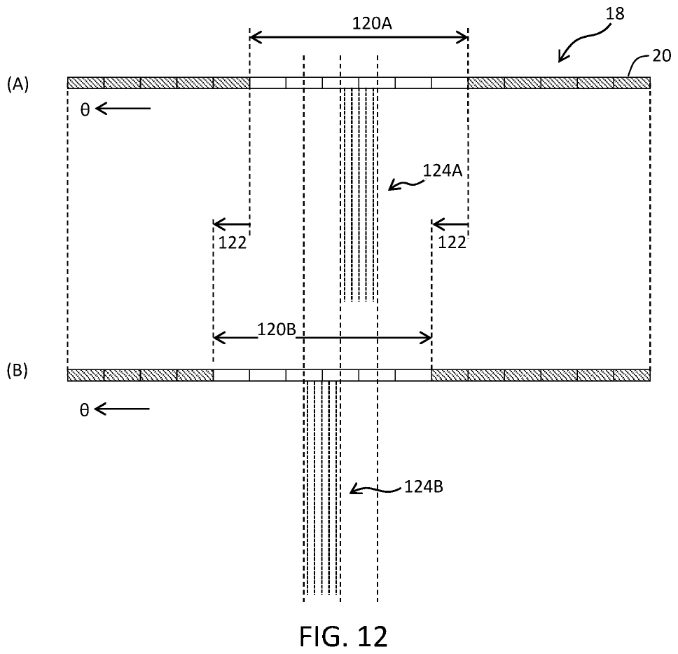

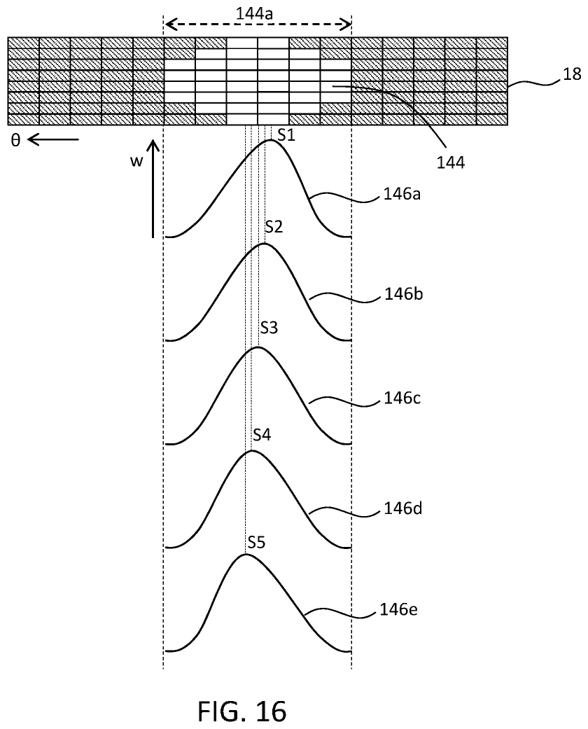

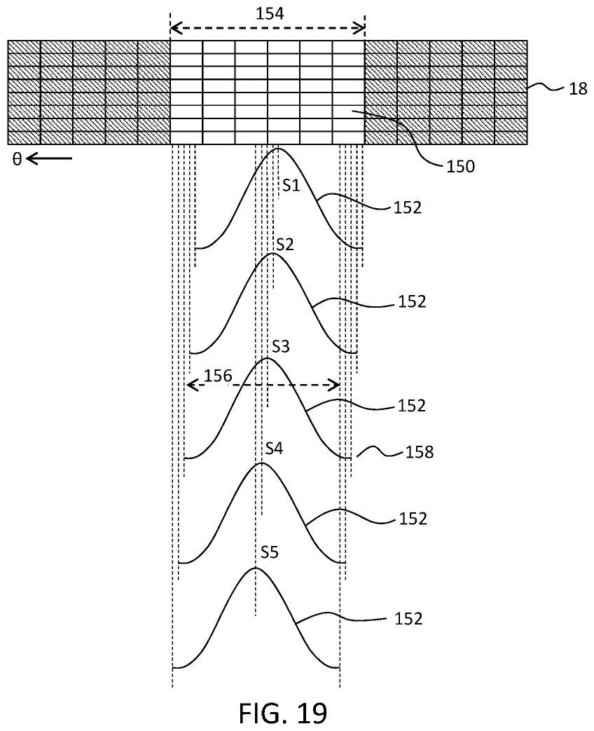

Incidentally, in the case of repeating transmission beam deflection scanning while sequentially changing from one opening position to another, when transmission voltage is equally applied to the whole of the transmission opening in the θ direction, inconsistency or steps may occur in transmission sound field before and after switching from one opening position to another, which may cause a vertical stripe pattern to be generated in an ultrasonic image. This problem will be described with reference to FIG. 12 to FIG. 15. Then, a solution for that problem will be described with reference to FIG. 16 to FIG. 18, and another solution will be described as a modification with reference to FIG. 19.

In FIG. 12, in (A), a transmission opening 120A set in a two-dimensional transducer element array 18 is shown. In (B), the next transmission opening 120B set in the two-dimensional transducer element array is shown. As already described, each of the transmission openings 120A and 120B is composed of a plurality of sub-arrays 20. A shift amount 122 between the transmission openings 120A and 120B corresponds to one sub-array 20. The transmission opening 120A is for performing transmission and reception with respect to a scanning line array 124A, and the transmission opening 120B is for performing transmission and reception with respect to a scanning line array 124B. The scanning line array 124A and the scanning line array 124B have a neighboring relation.

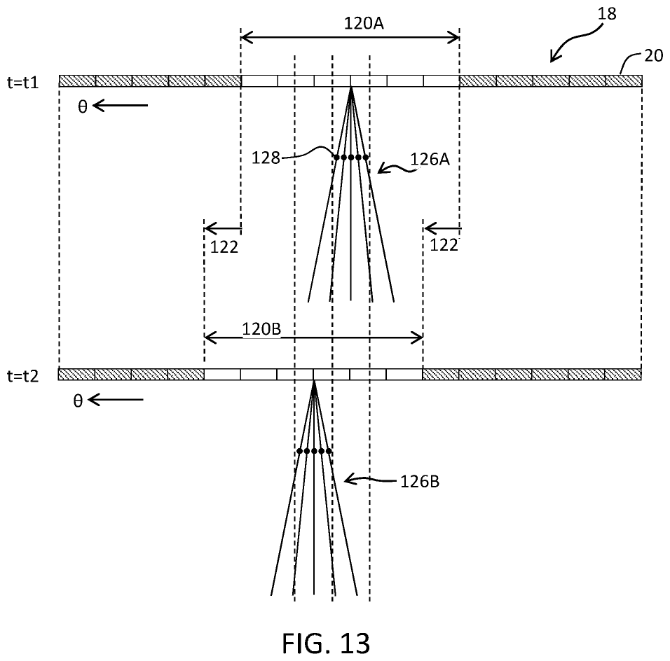

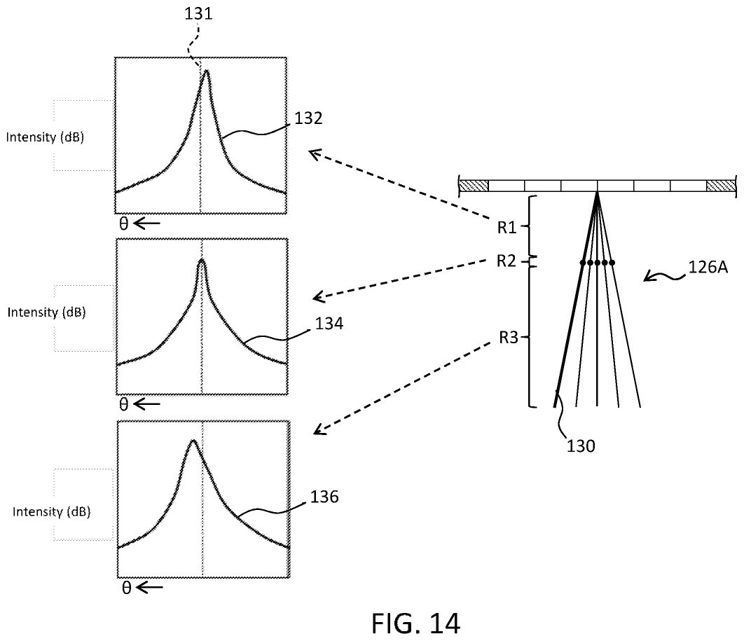

In FIG. 13, two transmission beam arrays 126A and 126B corresponding to the above-mentioned two scanning line arrays are shown. Reference symbol 128 indicates a transmission focus array. Here, as shown in FIG. 14, when attention is paid to a left-end transmission beam 130 of the transmission beam array 126A, in three sections R1, R2, and R3 in the depth direction, for example, three transmission beam profiles 132, 134, and 136 are observed. The horizontal axis of each of the transmission beam profiles 132, 134, and 136 corresponds to the θ direction, and the vertical axis thereof corresponds to transmission wave intensity. In the section R2 near the transmission focus, as shown in the transmission beam profile 134, the peak thereof coincides with a scanning line 131 corresponding to the transmission beam 130. Meanwhile, in the section R1 shallower than (in the front of) the transmission focus, as shown in the transmission beam profile 132, the peak thereof is deviated to the right from the scanning line 131. In the section R3 deeper than (in the rear of) the transmission focus, as shown in the transmission beam profile 136, the peak thereof is deviated to the left from the scanning line 131.

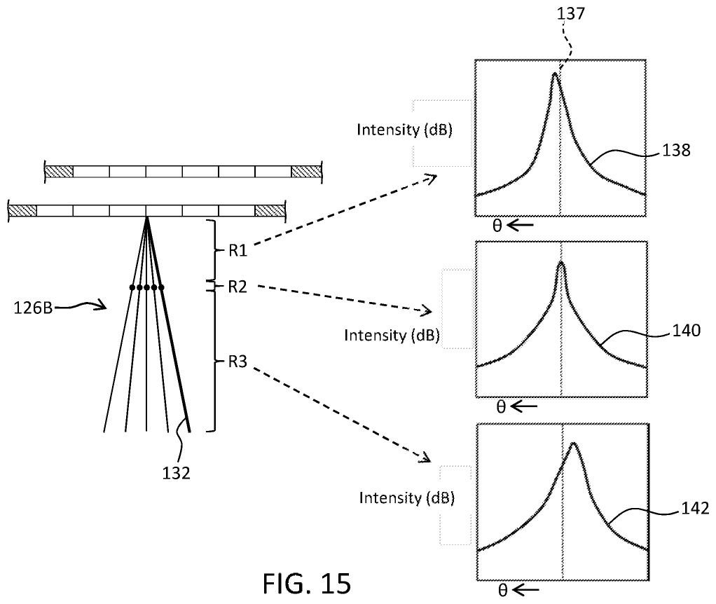

Subsequently, as shown in FIG. 15, when attention is paid to a right-end transmission beam 132 of the transmission beam array 126B which is formed using the next transmission opening, in three sections R1, R2, and R3 in the depth direction, for example, three transmission beam profiles 138, 140, and 142 are observed. Among them, in the transmission beam profile 140, the peak coincides with a scanning line 137 corresponding to the transmission beam 132; whereas in the transmission beam profile 138, the peak is deviated to the left from the scanning line 137, and in the transmission beam profile 142, the peak is deviated to the right from the scanning line 137. Before and after switching from one opening position to another, it is difficult to maintain a transmission sound field between two neighboring scanning lines constant, and due to this, vertical stripe patterns are likely to be generated in ultrasonic images.

In FIG. 16, a method for solving the above-mentioned problem is shown. In the example shown in the drawing, on the two-dimensional transducer element array 18, a transmission opening 144 having a polygonal shape or an ellipsoidal shape is set. The width (maximum width) of the transmission opening in the θ direction is denoted by reference symbol 144a. However, a transmission opening having a rectangular shape or any other shape may be set. With the opening position shown in the drawing, five scanning lines S1, S2, S3, S4, and S5 are associated.

In the case of forming a transmission beam with respect to the scanning line S1, a transmission apodization curve (a transmission weighting function) 146a is applied to the transmission opening 144. The horizontal axis of the transmission apodization curve corresponds to the θ direction, and the vertical axis thereof represents weight. As will be described below, to the y direction orthogonal to the θ direction, the same transmission apodization curve is commonly applied. In the case of forming transmission beams with respect to the scanning lines S2 to S5, transmission apodization curves 146b to 146e are applied to the transmission opening 144. The width of each of the transmission apodization curves 146a to 146e in the θ direction is the same as the width 144a of the transmission opening 144 in the θ direction. Also, five transmission focuses of the five transmission beams are set on the five scanning lines S1 to S5.

All of the forms of the transmission apodization curves 146a to 146e are generally mountain shapes; however, their vertex positions and their inclination directions are different from one another. Only the transmission apodization curve 146c has a bilaterally symmetric form, and the other transmission apodization curves 146a, 146b, 146d, and 146e have bilaterally asymmetric forms. Specifically, the vertex of the transmission apodization curve 146a is deviated to the right from the center in the θ direction, and the vertex coincides with the scanning line S1. The vertex of the transmission apodization curve 146b is deviated slightly to the right from the center in the θ direction, and the vertex coincides with the scanning line S2. The vertex of the transmission apodization curve 146c is at the center in the θ direction, and the vertex coincides with the scanning line S3. The vertex of the transmission apodization curve 146d is deviated slightly to the left from the center in the direction, and the vertex coincides with the scanning line S4. The vertex of the transmission apodization curve 146e is deviated to the left from the center in the θ direction, and the vertex coincides with the scanning line S5.

By applying the transmission apodization curves 146a to 146e as described above, in ranges on the individual scanning lines S1 to S5 between the side shallower than the transmission focus and the side far from the transmission focus (according to experiments, in most ranges except a very shallow part), it becomes possible to make the peaks of transmission beam profiles coincide with the scanning lines.

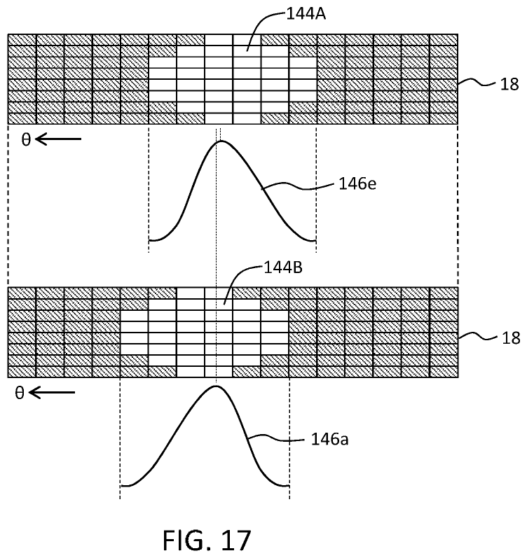

In FIG. 17, a transmission opening 144A and a transmission opening 144B neighboring each other are shown. In the case of forming a transmission beam corresponding to the left-end scanning line, using the transmission opening 144A, the transmission apodization curve 146e is applied. Next, the transmission opening 144B is selected, and in the case of forming a transmission beam corresponding to the right-end scanning line, using the selected transmission opening, the transmission apodization curve 146a is applied. As a result, before and after switching from one opening position to another, steps in the transmission sound field between two neighboring scanning lines are prevented or suppressed. Therefore, in an ultrasonic image, no vertical stripe pattern is generated.

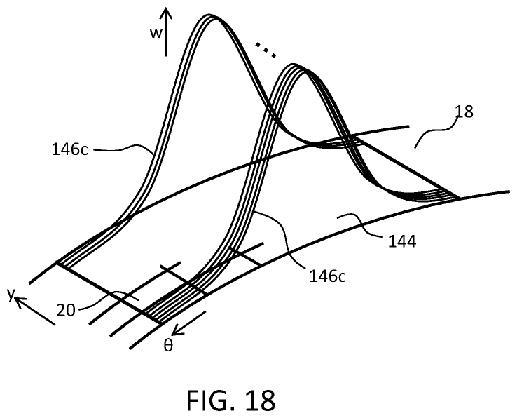

As shown in FIG. 18, in the two-dimensional transducer element array 18, the transmission opening 144 is composed of a plurality of transducer element rows arranged in the y direction. Each transducer element row is composed of a plurality of transducer elements arranged in the θ direction, and the number of transducer elements constituting each transducer element row depends on the shape of the transmission opening 144. Also, reference symbol 20 indicates a sub-array.

In the embodiment, as schematically shown in FIG. 18, to the plurality of transducer element rows constituting the transmission opening 144, the same transmission apodization curve is commonly applied. In FIG. 18, to the plurality of transducer element rows, the transmission apodization curve 146c is applied. Similarly to this, each of the other transmission apodization curves is commonly applied to the plurality of transducer element rows. By applying the same transmission apodization curve 146c to the plurality of transducer element rows, it is possible to suppress an increase in the amount of control for transmission apodization. Further, since it is possible to commonly apply the same transmission apodization curve array to the other transmission openings, in that sense, it is possible to suppress an increase in the amount of control. In FIG. 18, only the transmission apodization curve 146c is shown. However, the other transmission apodization curves 146a, 146b, 146d, and 146e (see FIG. 16) are also commonly applied to the plurality of transducer element rows.

Incidentally, since it is possible to individually invalidate sub-arrays 20 which do not constitute the transmission opening 144, it is not necessary to consider presence or absence of operation in sub-array units in transmission apodization control. In designing transmission apodization curves, a well-known β density function (see Patent Document 3) may be used. As a modification, there can be considered further commonly applying another transmission apodization curve array to a plurality of transducer element rows arranged in the θ direction (in that case, each transducer element row is composed of a plurality of transducer elements arranged in they direction). As a result, to each transducer element, a synthetic weight is applied. In general, transmission apodization can be performed in each transmission opening, in transducer element row units arranged in the y direction, or in transducer element row units arranged in the θ direction, or in transducer element row units arranged in the y direction and in transducer element row units arranged in the θ direction.

Scanning of the transmission openings in the θ direction is performed with the sub-array pitch, and this is rough control. Meanwhile, transmission beam deflection control and transmission apodization control in the θ direction are performed in transducer element units, and this is minute control. The configuration according to the embodiment is the combination of the rough control and the minute control in the θ direction. Therefore, it is possible to maintain or improve the qualities of ultrasonic images while reducing the amount of control.

In FIG. 19, another method for solving the problem which may occur before and after switching from one opening position to another is shown as a modification. In the two-dimensional transducer element array 18, a transmission opening 150 is set. In the example shown in the drawing, every sub-array in the transmission opening 150 is valid. When five transmission beams are formed corresponding to five scanning lines S1 to S5 by transmission beam deflection scanning, a transmission apodization curve 152 shown in FIG. 19 is applied. The width 156 of the transmission apodization curve 152 is smaller than the width 154 of the transmission opening 150 in the θ direction, and zero is set as the weight for a gap 158 between them. In other words, the width 156 defines an effective transmission opening in the θ direction. Incidentally, the transmission apodization curve 152 is commonly applied to the plurality of transducer element rows arranged in the y direction, as shown in FIG. 18.

In the procedure of sequentially forming five transmission beams corresponding to the scanning lines S1 to S5, the transmission apodization curve 152 is linearly scanned in the θ direction. The transmission apodization curve has a form bilaterally symmetric with respect to its peak. At each scanning position, the peak of the transmission apodization curve 152 coincides with a corresponding one of the scanning lines S1 to S5.

By such transmission apodization, over most of the depth range on each scanning line, it is possible to make the peak of the transmission beam profile coincide with the corresponding scanning line. As a result, it is possible to prevent or restrain steps from occurring in the transmission sound field before and after switching from one opening position to another.

According to the above-described embodiment, the transmission openings are configured in sub-array units, rather than in transducer element units, and the plurality of opening positions are determined with the sub-array pitch, rather than with the transducer element pitch. Therefore, it is possible to reduce the amount of control in scanning of each transmission opening. By reducing the amount of control, various advantages such as simplification of control, an increase in the control speed, a reduction in the size of the electronic circuit, a decrease in the power consumption of the electronic circuit, and a reduction in the cost are obtained. Also, according to the above-described embodiment, even if the plurality of opening positions are discretely set in the θ direction, since transmission beam deflection scanning is performed at each opening position, it is possible to avoid a decrease in scanning line density, or it is possible to realize a desired scanning line density. Therefore, an advantage that it is possible to prevent ultrasonic images from deteriorating in quality, or it is possible to improve the qualities of ultrasonic images is obtained. Further, according to the above-described embodiment, since it is possible to prevent or restrain steps in the transmission sound field from occurring before and after switching from one opening position to another, it is possible to prevent a deterioration in the qualities of ultrasonic images from being caused by a reduction in the amount of control.

1. An ultrasonic diagnostic device comprising:
a two-dimensional transducer element array that is composed of a plurality of sub-arrays arranged two-dimensionally;
an electronic circuit that is connected to the two-dimensional transducer element array and performs signal processing in sub-array units; and
a system controller that controls transmission and reception of ultrasonic waves by controlling the electronic circuit,
wherein by controlling the electronic circuit, a plurality of opening positions are determined on the two-dimensional transducer element array with a sub-array pitch along a scanning direction, at the plurality of opening positions two-dimensional transmission openings which are sub-array sets are sequentially set, and at each of the opening positions transmission beam deflection scanning in the scanning direction is performed.

2. The ultrasonic diagnostic device according to claim 1, wherein
the two-dimensional transducer element array is composed of a plurality of transducer elements arranged two-dimensionally along a convex surface having a curvature direction which is the scanning direction and having a width direction orthogonal to the curvature direction, and
the two-dimensional transmission opening is scanned in the curvature direction.

3. The ultrasonic diagnostic device according to claim 2, wherein
each of the sub-arrays has a longitudinal direction parallel with the curvature direction and a transverse direction parallel with the width direction, and in each of the sub-arrays, the number of transducer elements in the longitudinal direction is greater than the number of transducer elements in the transverse direction.

4. The ultrasonic diagnostic device according to claim 1, wherein
at each of the opening positions, a plurality of scanning lines which spread out radially from an origin point are determined,
at each of the opening positions, a plurality of transmission beams which spread out radially from the center of the two-dimensional transmission opening are formed, and
a plurality of transmission focuses are formed on the plurality of scanning lines.

5. The ultrasonic diagnostic device according to claim 4, wherein
according to the deflection angle of each of the transmission beams, a transmission apodization curve to be used is selected from a transmission apodization curve array.

6. The ultrasonic diagnostic device according to claim 5, wherein
the transmission apodization curve array is commonly used for the plurality of opening positions.

7. The ultrasonic diagnostic device according to claim 5, wherein
each of the transmission apodization curves has a form for making a peak of a profile of each transmission beam coincide with each scanning line in the front and rear of a transmission focus on the corresponding scanning line.

8. The ultrasonic diagnostic device according to claim 5, wherein
each two-dimensional transmission opening is composed of a plurality of transducer element rows arranged in a direction orthogonal to the scanning direction,
each transducer element row is composed of a plurality of transducer elements arranged in the scanning direction, and
each transmission apodization curve is commonly applied to the plurality of transducer element rows arranged in the orthogonal direction.

9. The ultrasonic diagnostic device according to claim 5, wherein
the electronic circuit includes a plurality of transceivers connected to the plurality of transducer elements constituting the two-dimensional transducer element array,
each transceiver includes a transmission voltage generation circuit configured to generate transmission voltage which is defined by the transmission apodization curve which is used,
each transmission voltage generation circuit generates the transmission voltage by dividing a maximum transmission voltage, and
a voltage control value standardized according to the maximum transmission voltage is given to each transmission voltage generation circuit.

10. The ultrasonic diagnostic device according to claim 2, wherein
the shape of a two-dimensional transmission opening which is set at each opening position is a polygonal shape which is formed by cutting off four corners from a rectangular shape extending in the curvature direction, or an ellipsoidal shape extending in the curvature direction.

11. The ultrasonic diagnostic device according to claim 1, wherein
during transmission beam deflection scanning at each opening position, in the two-dimensional transmission opening, a transmission apodization curve is scanned in the scanning direction while its shape is maintained.

12. A transmission control method of an ultrasonic diagnostic device, wherein
by controlling an electronic circuit connected to a two-dimensional transducer element array composed of a plurality of sub-arrays arranged two-dimensionally,
a plurality of opening positions are determined on the two-dimensional transducer element array, with a sub-array pitch along a scanning direction,
at the plurality of opening positions, two-dimensional transmission openings which are sub-array sets are sequentially set, and
at each of the opening positions, transmission beam deflection scanning in the scanning direction is performed.
